(19) United States
(12) Patent Application Publication
(10) Pub. No.: US 2021/0338803 A1
(43) Pub. Date: Nov. 4, 2021

(54) DUAL-MOLECULAR DNA VACCINE COMPOSED OF A VIRAL ANTIGEN AND AN IMMUNE COSTIMULATOR

(71) Applicant: SICHUAN CHENGYU BIOLOGICAL PRODUCTS INC., Chengdu (CN)

(72) Inventors: Li YU, Gaithersburg, MD (US); Zhen ZENG, Chengdu (CN); Lian ZENG, Chengdu (CN); Mei ZHANG, Chengdu (CN)

(21) Appl. No.: 16/874,431

(22) Filed: May 14, 2020

Related U.S. Application Data
(63) Continuation of application No. PCT/CN2020/087720, filed on Apr. 29, 2020.

(51) Int. Cl. A61K 39/215; A61K 39/39

(52) U.S. Cl. CPC A61K 39/215; A61K 2039/53; A61K 39/39

(57) ABSTRACT

The present invention discloses a dual-molecular DNA vaccine composed of viral antigen and immunization coactivator, which relates to the technical field of biomedicine. The said vector is a DNA plasmid; the viral antigen is any viral immunogenic (antigen) molecule; the immunological activator is a kind of T cell costimulators. The vaccine disclosed in the present invention is that two gene fragments expressing the T cell costimulator and the viral antigen molecule are constructed into one plasmid DNA and will be co-expressed in a cell at the same time to activate the systemic immune response through the two signalings. The dual-molecular DNA vaccine also is a novel platform of vaccine technology, using for development of multiple vaccines to prevention from various infectious diseases. Especially, a dual-molecular DNA vaccine with the SARS-CoV-2 S antigen and T cell costimulator can be constructed through this technology platform to prevent and control global pandemic COVID-19.

### CROSS-REFERENCE TO RELATED APPLICATION

This application is a continuation of International Application No. PCT/CN2020/087720, filed on Apr. 29, 2020, the content of which is incorporated herein by reference in its entirety.

### TECHNICAL FIELD

The present invention relates to the technical field of biomedicine, in particular to a type of DNA vaccine of antigen and immune co-activating factors used for the prevention of infectious diseases.

### BACKGROUND

The coronavirus pneumonia (COVID-19) is a new type of infectious disease caused by a novel coronavirus (SARS-CoV-2). On Mar. 11, 2020, the World Health Organization announced that the COVID-19 was a pandemic. So far, the coronavirus pandemic has caused more than 3 million cases and more than 200,000 died worldwide, and led to the downside of the global economy. The prevention and control of pandemic are very important. In order to stop the disease epidemic, it is imminent to develop and to produce fast and effective vaccines to help quick population immunity.

However, the traditional vaccine development relies on screening of attenuated virus strain through generations of passage to passage, which would take a longer time to obtain. Another bottleneck for the COVID-19 vaccine is that there is no sensitive animal available, which makes it more difficult to screen and identify the selected attenuated vaccine strains. Yet, the development and screening of such a highly infectious virus vaccine require safer manufacturing, such as P3 and P4 conditions. However, at present, there are a few laboratories that meet the safety requirements. Therefore, it is not difficult to understand that the traditional vaccine development needs several years and it is hard to become the savior as we are looking for since the development of the vaccine is urgent.

As well-known, antigenicity is one of the main factors that determine the specificity and effectiveness of a vaccine. The modern genetic engineering makes vaccine development no longer rely on traditional methods, while genetic engineering vaccine can greatly shorten the developing time. Genetic engineering vaccines usually include DNA or RNA vaccines. Compared with methods to develop the traditional vaccine, genetic engineering vaccine has many potential advantages, including stimulating B cell and T cell responses, improving the stability of the vaccine. The main advantage of genetic engineering vaccines is safer. It does not need to deal with toxic pathogens or use pathogens for production purposes to quickly produce new vaccines. In the past 30 years, many clinical studies have not reported that the use of genetic engineering vaccine caused adverse or toxic reactions.

DNA vaccines express antigens in cells in the body to activate the immune response to microorganisms. DNA vaccine can not only be produced massively in E. coli, but also be cost less and easy to store and transport. In addition, the DNA vaccines can greatly shorten the time of vaccine development. Its main advantages are good safety for not need to deal with toxic pathogens, but the rapid production of new vaccines. DNA vaccines use DNA plasmids as carriers to express specific antigen to activate the immune system. However, the limited antigen expression of DNA vaccine in cells is not enough to effectively stimulate the immune response to the antigen. For this reason, so far, there is no DNA vaccine that has been approved for inoculation to humans.

Because low immunogenicity has always been a major obstacle to the use of DNA vaccine inoculation in humans, scientists have taken various steps to increase the intensity and duration of DNA vaccine to induce the immune response. One strategy is to create vaccine mixtures, which include DNA vaccines and plasmids encoding immune regulatory proteins (such as IL-2, IL-12, etc.), plasmids activating and/or enhancing APC activity (GM-CSF), or CXC chemokines (IL-8), and injection of CC chemokines (such as macrophage inflammatory protein MIP-1 α, mip-3 β). In addition, the immunogenicity of the DNA vaccine can also be enhanced by additional DNA plasmid encoding costimulatory molecules and adhesion molecules. The common application of all these DNA vaccines+immunomodulators proves that the reasonably designed DNA vaccine and immunotherapy can greatly improve the immunogenicity of DNA vaccine and thus increase the efficiency of DNA vaccination.

Although a variety of different immune regulatory factors have been used in DNA vaccines, they have mainly constructed alone and separated from the DNA plasmids expressing the antigen and the two DNA plasmids are mixed for local administration. The mixed DNA vaccines cannot effectively present the two signals to immune cells, so the induction of immune response to specific antigens cannot be efficient.

### SUMMARY

In order to overcome technical hurdles in DNA vaccine, the invention discloses a dual-molecular DNA vaccine composed of a virus antigen and an immune costimulator, which can make the threshold of immunogenicity lower and thus raise the vaccine capability of the immune responses (FIG. 1).

The dural-molecular DNA vaccine holds an antigen segment and human immune co-stimulatory genes within a DNA plasmid and thus expresses the two signals within a cell to specifically activating the immune responses to the virus pathogen and protecting from the virus infection.

To achieve the purpose of this invention, the present invention provides the following technical solutions.

A dual-molecular DNA vaccine of viral antigen-immune costimulator comprises an expression vector and the gene fragments of a viral antigen and a human T cell costimulator loaded on the same expression vector. The expression vector is a DNA plasmid; the viral antigen gene can be any virus-specific antigen gene fragment; the immunological activator gene encodes for a T cell costimulator.

In the said technical solution, the dual-molecule DNA vaccine uses a two signaling that is a co-stimulation through both a human immunogenic factor and a viral antigen expressed in the same cell to activate the immune system, specifically activating the system immune response to prevent from the viral infection. The immune gene DNA vaccine also can be used as a technical platform for dual-molecular expression and thus enhance the activation of the immune system. The platform of dual-molecular DNA vaccines can be used to construct other DNA vaccines carrying specific pathogenic antigen. It is suitable for the development and construction of preventive vaccines for a variety of infectious diseases, including global pandemic COVID-19. A dual-molecular DNA vaccine containing the S antigen of a SARS-CoV-2 and immune costimulator can be created through the technology platform.

In the said technical solution, the gene fragments of the human immunological factor and a viral antigen molecule are inserted at the downstream of a promoter in the DNA plasmid through genetic engineering. Both then can be transcribed and expressed in a non-fusion form within a cell. The two molecules (two signals) are co-expressed in the same cell thus specifically enhance the activation of systemic immunity to the virus.

Further, the said gene fragments of human immunological factor expressed in B cells or in antigen presenting cells are T cell costimulatory genes.

Further, the said T cell costimulator can specifically activate different types of T cell subsets; the T cell costimulators include: any one of the CD80, CD86, ICOSL, OX40L, CD40, 4-1BBL, CD70, CD30L, CD48.

Further, the T cell subsets include any of CD4 cells, CD8 cells, NK cells, cytotoxic T cells, lymphokine T cells, inducible T cells, and any helper T cells.

Further, the said human T cell costimulator can be expressed in the functional forms of either homologous or heterologous fusion molecules.

Further, the human T cell costimulator can be expressed as a functional protein or a protein polypeptide in the cell.

The functional activity of the human T cell costimulator provides the necessary second signal for activating the T cells and enhances the specific systemic immune response to the co-expressed antigen.

Further, the said protein polypeptide is a modified or mutated active protein polypeptide.

Further, the viral antigen molecule and the human T cell costimulator encoded in a DNA plasmid are simultaneously expressed in a non-fusion form of two molecules in a cell.

The invention also discloses the application method of the dual-molecular DNA vaccine. The structure of the dual-molecular DNA vaccine is a DNA biological macromolecule, which is inoculated in the body in the form of a DNA plasmid for preventing and controlling various infectious diseases.

Preferably, the dual-molecular DNA vaccination can be composed of any virus-specific antigen and one or more of the several human T cell costimulators, which are formulated in a treatment course to maximize the activation of the immune system.

The present invention uses a DNA plasmid containing an antigen and a T cell costimulator as a technical platform for immune vaccines, in which any specific viral antigen gene can be inserted into this dual-molecular DNA plasmid (vaccine) to enhanced specific immune response. Therefore, different immune DNA vaccines can be obtained by using different specific antigen genes.

### BRIEF DESCRIPTION OF THE DRAWINGS

The following drawings are merely examples for illustrative purposes according to various disclosed embodiments and are not intended to limit the scope of the present disclosure.

FIG. 1. A schematic diagram of the effective activation of the immune system depends on antigenicity and antigen concentration. The dual-molecular DNA vaccines can reduce the threshold of immunogenicity and thus raise the capability of the immune response.

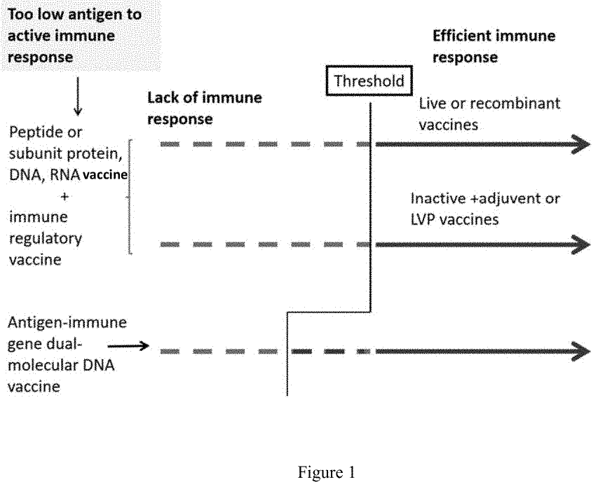

FIG. 2. A schematic diagram of a DNA plasmid carrying the SARS-CoV-2 S antigen gene and a human T cell costimulator described in the present invention.

FIG. 3. Immune fluorescent assay results of co-expression of the SARS-CoV-2 S antigen and the T cell costimulator by the dual-molecular DNA plasmid in cells.

### DETAILED DESCRIPTION OF THE PREFERRED EMBODIMENT

In order to clearly describe the purpose, technical solutions, and advantages of the present invention, the following is a detailed description of the present application accompanying the drawings and embodiments. It should be understood that the specific embodiments described herein are only used to explain the present application and are not used to limit the present application.

The expression site and function of human T cell costimulators in this invention are shown in patent CN2019111493147 and thus will not be listed in detail in this application.

The viral antigen molecule is the S antigen of SARS-CoV-2. The gene fragments of SARS-CoV-2 S antigen and the human T cell costimulator were constructed in a DNA plasmid to obtain a dual-molecular vaccine that uses for the prevention of COVID-19.

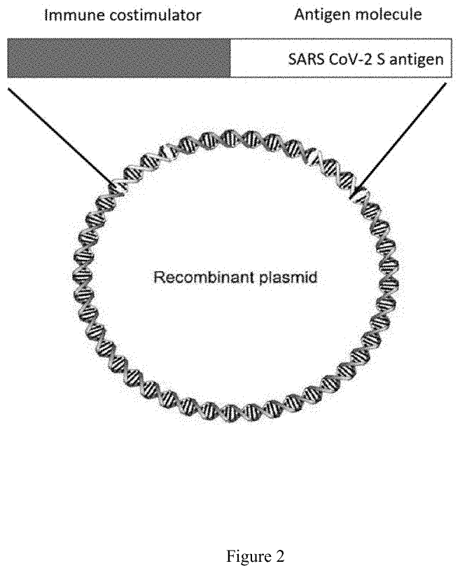

As shown in FIG. 2, in the following example, the expression vector is a DNA plasmid, in which the gene fragments of human T cell costimulators and the SARS-CoV-2 S antigen (GenBank: MN988713.1) were inserted through conventional genetic engineering.

The construction and detection methods for dual-molecular DNA vaccine are as follows:

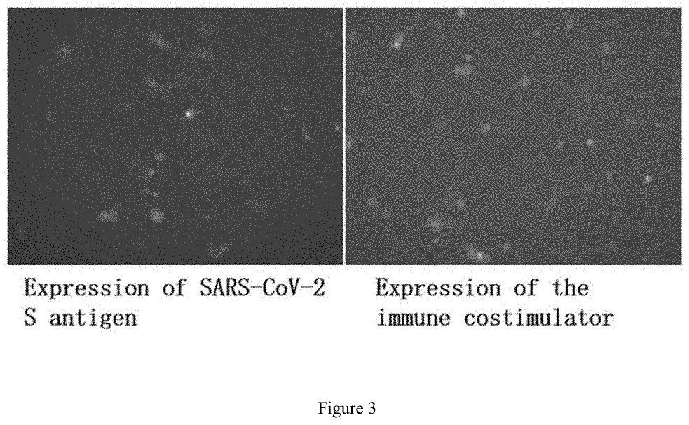

the gene fragments of human or murine T cell costimulator and the SARS-CoV-2 S antigen or a GFP were PCR synthesized and connected to the downstream of a promoter in DNA plasmid vector with the same sites of restriction enzymes (FIG. 2). Positive clones were screened by enzyme digestion, sequencing analysis, and by the expression of the inserted foreign gene in transfected cells through immunofluorescence assay (FIG. 3).

The present invention is not limited to the foregoing specific embodiments. The invention extends to any new feature or any new combination disclosed in this specification, as well as any new method or process step disclosed or any new combination.

What is claimed is:

1. A dual-molecular DNA vaccine of viral antigen-immune costimulator is comprised of an expression vector, carrying the gene fragments of a viral antigen and human immune activator, of which the expression vector is a DNA plasmid; the said viral antigen is any viral-specific antigen molecule; the immune activator is the T cell costimulators.

2. The dual-molecular DNA vaccine of virus antigen-immune costimulator of claim 1, wherein, the said T cell costimulators are expressed originally in B cells or antigen-presenting cells as second signaling necessary to activate T cells.

3. The dual-molecular DNA vaccine of virus antigen-immune costimulator of claim 1, wherein, the T cell costimulators include any one of CD80, CD86, Icosl, OX40L, CD40, 4-1BBL, CD70, CD30L, and CD48.

4. The dual-molecular DNA vaccine of virus antigen-immune costimulator of claim 1, wherein, the T cell costimulators can specifically activate different types of T cell subsets, including CD4 cells, CD8 cells, NK cells, cytotoxic T cells, lymphokine T cells, inducible T cells, and helper T cells.

5. The dual-molecular DNA vaccine of virus antigen-immune costimulators of claim 3, wherein, any one of the T cell costimulators are expressed in cells as one or more homologous or heterogenous fusion molecules.

6. The dual-molecular DNA vaccine of virus antigen-immune costimulators of claim 3, wherein, the T cell costimulators are expressed as a functional protein or a functional protein polypeptide in cells.

7. The dual-molecular DNA vaccine of virus antigen-immune costimulators of claim 3, wherein, the T cell costimulators are expressed to be an active protein or a modified or mutated active polypeptide.

8. The dual-molecular DNA vaccine of virus antigen-immune costimulators of claim 1, wherein, the said gene fragments of viral antigen and human T cell costimulator are constructed in a DNA plasmid and expressed simultaneously to be a non-fusion form of two protein molecules.

9. The dual-molecular DNA vaccine of virus antigen-immune costimulators of claim 1, wherein, the said any viral protein molecule with antigenicity can be integrated as a molecule together with the human T cell costimulator to create a dual-molecular DNA vaccine.

10. The dual-molecular DNA vaccine of virus antigen-immune costimulators of claim 9, wherein, the virus antigen molecule can be the spike protein (antigen) of the SARS-CoV-2.


SEQUENCE LISTING (9 sequences)

SEQ ID NO: 1 - protein, 1273 aa, Homo sapiens
  MISC_FEATURE (1)..(1273): SARS-Cov2 antigen
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 329 aa, Homo sapiens
  MISC_FEATURE (1)..(329): CD86 T cell costimulator
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 3 - protein, 302 aa, Homo sapiens
  MISC_FEATURE (1)..(302): CD80 T cell costimulator
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 4 - protein, 199 aa, Homo sapiens
  MISC_FEATURE (1)..(199): OX40L T cell costimulator
MEGEGVQPPDENLENGSRPRFKWKKVLRLVVSGIKAAGLLLCVVYVCLQFSSSPAKDSPIQRLRAPVTGCEGGRLFIGTSKNEYETMEVQNNSVIINCDGLYLIHLKGSFFQEVKINLHFRKDRSPIFVPMLNNGQRVVFTVVTSLAFKDEVYLTVNASDTLCEHLQINDGELIIVQLTPNGYCAPERPYSSTVNQVPL

SEQ ID NO: 5 - protein, 277 aa, Homo sapiens
  MISC_FEATURE (1)..(227): CD40 T cell costimulator
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 6 - protein, 254 aa, Homo sapiens
  MISC_FEATURE (1)..(254): 4-1BBL T cell costimulator
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 7 - protein, 193 aa, Homo sapiens
  MISC_FEATURE (1)..(193): CD70 T cell costimulator
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 8 - protein, 234 aa, Homo sapiens
  MISC_FEATURE (1)..(234): CD30L T cell costimulator
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 9 - protein, 257 aa, Homo sapiens
  MISC_FEATURE (1)..(257): CD40 T cell costimulator
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSFGVEWIASWLVVTVPTILGLLLTATTRNEYDCKETNS